(19) United States
(12) Patent Application Publication
(10) Pub. No.: US 2025/0073329 A1
(43) Pub. Date: Mar. 6, 2025

(54) PRODUCTION OF ELISA REAGENTS AND VACCINES BY 'ONE PROCESS'

(71) Applicant: KING ABDULAZIZ UNIVERSITY, Jeddah (SA)

(72) Inventors: Taha KUMOSANI, Jeddah (SA); Elie BARBOUR, Beirut (LB); Werner KRULL, Nordhorn (DE); Soonham YAGHMOOR, Jeddah (SA)

(21) Appl. No.: 18/461,835

(22) Filed: Sep. 6, 2023

(51) Int. Cl. A61K 39/215; G01N 33/543

(52) U.S. Cl. CPC A61K 39/215; G01N 33/54306; G01N 2333/165

(57) ABSTRACT

The invention generally relates to a novel ‘One Process’ for production of polypeptides comprising an anti-idiotypic, mirror image of the antigenic determinants of an agent of interest such as a disease-causing agent. In particular, the polypeptides are used to pre-treat samples prior to indirect ELISA assays and in its incorporation in vaccines. When used in indirect ELISA assays, the result is a significant reduction in false positives, while its inclusion in vaccines provides an additive induction of immunity in humans and different animal species by the proteinaceous mirror image of weak immunogens including, lipid, carbohydrate, and low molecular weight molecules of the agent of interest. The incorporation of anti-idiotypes produced by the ‘One Process’ in vaccines induces antibodies that is used in cocktail of therapeutics for treatment of injurious antigens and autoimmune reactions in humans and animals.

### BACKGROUND OF THE INVENTION

### Field of the Invention

The invention generally relates to a novel ‘One Process’ for production of polypeptides comprising an anti-idiotypic, mirror image of the antigenic determinants of an agent of interest such as a disease-causing agent. In particular, the polypeptides are used to pre-treat samples prior to indirect ELISA assays and in its incorporation in vaccines. When used in indirect ELISA assays, the result is a significant reduction in false positives, while its inclusion in vaccines provides an additive induction of immunity in humans and different animal species by the proteinaceous mirror image of weak immunogens including, lipid, carbohydrate, and low molecular weight molecules of the agent of interest. The incorporation of anti-idiotypes produced by the ‘One Process’ in vaccines induces antibodies that is used also in cocktails of therapeutics for treatment of injurious antigens and autoimmune reactions in humans and animals.

### Description of Related Art

In most direct and indirect Enzyme-Linked Immunosorbent Assay (ELISA) assays which target diagnosis and control of animal and human diseases, cross-reactivity is the cause of false positivity in generated data. This lowers the significance of differences between negative and positive samples, thus affecting the data's interpretation and the implementation of sound control programs. In 2008, the CDC issued warnings about the public health consequences of false-positive laboratory test results for Brucella. Magtoto, et. al, 2019, evaluated and confirmed serologic cross-reactivity between transmissible gastroenteritis coronavirus and porcine respiratory coronavirus using commercial ELISA kits. Moreover, Kam et al., 2015 studied the cross-reactivity of chikungunya virus specific anti-E2EP3 antibodies in arbovirus-infected patients, and confirmed it. Guerrieri et al., 2019 investigated the detecting capabilities of the immune analysis of direct ELISA kits against fentanyl in whole blood and found cross-reactivity with several fentanyl analogs (2-fluorofentanyl, acetylfentanyl, acrylfentanyl, carfentanil, cyclopropylfentanyl, tetrahydrofuranylfentanyl, furanylfentanyl, ocfentanil, valerylfentanyl).

Currently, due to the ongoing pandemic, many scientists are focusing on diagnosis and control programs of COVID-19 which is caused by SARS-COV-2. The emergence of this new β-coronavirus outbreak represents a challenge for development of diagnostic kits to evaluate the dynamics of humoral immunity post vaccination or infection (indirect ELISA), with difficulty in satisfying the requirements for elimination of cross-reactivity by antibodies that are acquired due to infection by other prevalent human Coronaviruses (CoVs). This includes CoVs that cause the common cold around the globe namely, types 229E, NL63, OC43, and HKU1, as well as zoonotic and highly pathogenic CoVs, including SARS-COV-1 and MERS-CoV.

There is an urgent and ongoing need to develop methods and tests with low levels of false positives in order to accurately detect antibodies to agents of interest, especially infectious agents such as viruses.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

The present disclosure describes a ‘One Process’ method that produces anti-idiotype reagents for i) the elimination of cross-reactivity in indirect ELISA kits, and ii) for use as vaccines. Each anti-idiotype is a polypeptide mirror image of the antigenic determinants of an agent of interest, such as a disease-causing agent, and generally, any molecule agent that is capable of inducing an immunity, specially called an immunogen. The design of each anti-idiotype is such that, when used alone or together with other anti-idiotypic polypeptides, they overcome the low immunogenicity often exhibited e.g. by the lipid bilayer of the enveloped viruses, carbohydrate capsules of bacterial etiology, and any other weak immunogens. When used as part of an indirect ELISA test, the anti-idiotype reagents can greatly reduce or eliminate false positives. When used as a vaccine, the anti-idiotype reagents elicit a robust immune response. In some aspects, the agent of interest is a coronavirus, such as a SARS-Cov-2 variant.

It is an object of this invention to provide a ‘One Process’ method for producing anti-idiotypes, that will be applicable on any commercial or underdeveloped indirect ELISA, by contacting the samples with the produced anti-idiotypes to eliminate the cross-reacting idiotypes of different isotypes, leading to higher specificity in detection and/or quantitation of antibodies to the targeted antigen. The step of contacting causes the at least one anti-idiotypic polypeptide to bind to the cross-reactive antibody and form an anti-idiotypic polypeptide-antibody complex, followed by removing the anti-idiotypic polypeptide-antibody complex from the sample; and conducting the indirect ELISA assay to detect the antibodies to the one or more epitopes on the targeted antigen. The antigens include those collected from humans, animals, plants, and the environment. In some aspects, the at least one anti-idiotypic polypeptide, produced by the ‘One Process’, is immobilized on a support. In further aspects, the support is a bead. In additional aspects, the at least one anti-idiotypic polypeptide, produced by the ‘One Process’ is reacted with the sample in its liquid phase without an immobilization of the anti-idiotypic polypeptide on a solid support, resulting in a precipitate that is removed by centrifugation. In aspects, of the invention, the targeted antigen is presented on a virus, bacteria, protozoa, fungi, allergen, toxin, and other free or conjugated molecules. In some aspects, the virus is a coronavirus. In further aspects, the cross-reacting coronavirus is selected from the group consisting of HCoV-HKU1, HCoV-229E, HCoV-NL63, HCoV-OC43, SARS-COV-1, and MERS-COV, while the qualitative and quantitative indirect ELISA targets the assessment of antibodies, qualitatively or quantitatively, to SARS-Cov-2 antigen(s).

Also provided is a composition of anti-idiotypic polypeptides produced by the ‘One Process’ comprising of at least one anti-idiotypic polypeptide, which is specific to a variable region of an antibody combining site (ACS) of one or more non-specific isotypes that cross-react against coronavirus epitope(s). In some aspects, the coronavirus is SARS-COV-2 or other CoV types.

A ‘One Process’ method of producing anti-idiotypes for elimination of cross reactivity in indirect ELISA, will include the anti-idiotypes, singly or in different combinations, in vaccines, since they are mirror images of antigens carried on different types of coronaviruses, resulting in vaccines for the control of HCoV-HKU1, HCOV-229E, HCoV-NL63, HCoV-OC43, SARS-COV-1, SARS-COV-2 and MERS-COV. In some aspects, the ‘One Process’ method produces anti-idiotypes that are mirror images of any etiologic agent, a microbe or a molecule, involved in human diseases and veterinary diseases of different domestic and wild species. The ‘One Process’ method produces anti-idiotypes, mirror images of etiologic agents, to include the anti-idiotypes in vaccines to acquire immunity against viruses, bacteria, protozoa, fungi, toxins, allergens, tumor antigens, and specific harmful idiotypes in autoimmunity diseases of humans and animals. In some aspects, the ‘One Process’ method produces anti-idiotypes, mirror images of etiologic agents, to include the anti-idiotypes in vaccines to acquire therapeutic cocktail of antibodies that are specific to epitopes on harmful antigens that are causing injuries in humans and in different animal species. In further aspects, the ‘One Process’ method produces anti-idiotypes, to include in vaccines to produce therapeutics for reacting against the paratope of antibodies that are involved in autoimmune diseases, due to their binding to self-antigens in the body of humans and animals.

The therapeutics produced in the ‘One Process’ against paratopes of antibodies causing autoimmune diseases includes the following harmful antibodies namely, anti-actin, anti-centromere, anti-ganglioside, anti-mitochondria, anti-neutrophils, anti-nuclear, anti-signal recognition peptide, anti-smooth muscle, anti-glomerular basement membrane, anti-parietal cell, anti-liver-kidney microsomes, anti-soluble liver antigen, etc.

The objective of this invention is to present a ‘One Process’ method for producing anti-idiotypes, that will be applied on any commercial or underdeveloped indirect ELISA, by contacting the samples with the produced anti-idiotypes to eliminate the cross-reacting idiotypes of different isotypes, leading to higher specificity in detection and/or quantitation of antibodies to the targeted antigen, wherein the step of contacting causes the at least one anti-idiotypic polypeptide to bind to the cross-reactive antibody and form an anti-idiotypic polypeptide-antibody complex, followed by removing the anti-idiotypic polypeptide-antibody complex from the sample; and conducting the indirect ELISA assay to detect and/or quantify the antibodies to the one or more epitopes on the targeted antigen. The antigens of interest include viruses, bacteria, protozoa, fungi, allergens, toxins, polymers, and any other molecule that is an immunogen. These antigens are collected from humans, animals, plants, and environment.

Example of cross-reaction elimination from indirect ELISA that detects and quantify antibodies specific to SARS-COV-2, the causative virus of COVID-19, is by reacting the liquid samples with six anti-idiotypes that will lead to elimination of antibodies against six respective human coronaviruses namely, HCoV-HKU1, HCOV-229E, HCoV-NL63, HCoV-OC43, and MERS-COV.

The idiotypes produced by the ‘One Process’ for elimination of cross-reactivity to the antigen of interest (SARS-COV-2), will be incorporated individually, or collectively in vaccines, as mirror image(s) of the human Coronavirus(s).

The invented ‘One Process’ could be used to produce anti-idiotypes for eliminating cross-reactions to any other antigen of interest, and to incorporate the produced anti-idiotypes in vaccine and other therapeutic manufacturing for human and animal use.

### DETAILED DESCRIPTION

The present disclosure provides a protocol for manufacturing a wide spectrum of anti-idiotypes (e.g. anti-idiotypic antibodies, anti-idiotypic polypeptides, anti-idiotype reagents, etc.) that solve the problem of false positives in any indirect ELISA, and the inclusion of such anti-idiotypic polypeptides in developing vaccines and antibody therapeutic reagents for human and animal use.

SARS-COV-2 was used as an exemplary prototype for development of the technology. As discussed in detail below, the methodology significantly increased the specificity of manufactured commercial indirect ELISAs to SARS-COV-2, the etiologic agent of the COVID-19 pandemic. Moreover, the anti-idiotypes of the different CoV types, and their use as safe and efficient vaccines, was confirmed by reacting anti-idiotypic polypeptides, mirror images of their respective CoV types, with seroconverted polyvalent antibodies acquired in humans that were CoV positive by PCR.

### Definitions

Idiotype: in immunology, an idiotype is a shared characteristic between a group of immunoglobulin or T-cell receptor (TCR) molecules, based upon the antigen binding specificity and therefore structure of their variable region. The variable region of antigen receptors of T cells (TCRs) and B cells (immunoglobulins) contain complementarity-determining regions (CDRs) with unique amino acid sequences. They define the surface and properties of the variable region, determining the antigen specificity and therefore the idiotope of the molecule. Antibody idiotype is determined by gene rearrangement, junctional diversity, P-nucleotides (palindromic nucleotides at sites of single-strand breaks), N-nucleotides, and/r somatic hyper-mutations.

Anti-idiotype: an antibody that binds to the antigen combining site of another antibody either suppressing or enhancing the immune response. Anti-idiotypes are secondary antibodies acquired by the immune system in reaction to antibodies from a different species of animal. Indirect ELISA is a two-step ELISA which involves the binding of a primary antibody to the antigen of interest present on a solid matrix and binding of a labeled secondary antibody (conjugate) to the light and/or the heavy chains of the primary antibody. The primary antibody is incubated with the antigen, followed by the incubation with a labelled secondary antibody that exerts its enzymatic activity on a substrate, to result in a colorimetric system, in which its optical density is positively correlated to the concentration of the primary antibody present in the sample.

### Production of an Anti-Idiotype Reagent

The anti-idiotype reagents disclosed herein are generally polypeptides that are anti-idiotype antibodies or portions thereof that retain a desired activity, such as the ability to bind to cross-reactive antibodies and reduce or eliminate false positive reactions in Indirect-ELISA assays, and/or to elicit an immune response in a vaccine, replacing weak immunogens such as the lipid and carbohydrate antigenic determinants, or any other molecules of low molecular weights, such as haptens. To make the anti-idiotype polypeptides of the invention, a practitioner performs the following steps:
1) Identifying at least one antigen of interest which may be present on an agent of interest, such as an infectious agent. As described in detail below, a wide variety of agents and antigens may be targeted.
2) Raising first antibodies in a first host species to the at least one antigen of interest. Each first antibody contains an antigen-combining site (paratope) that binds directly to the antigen of interest to which it was raised against. As is known in the art, a paratope is the part of an antibody which recognizes and binds to an epitope on the antigen. It is a small region at the tip of the antibody's antigen-binding fragment and contains variable parts of the antibody's heavy and light chains.
3) Raising second antibodies in a different host species to the first antibodies. Each second or “anti-idiotypic” antibody contains a binding site that binds directly to the antigen-combining site (paratope) of a first antibody.

The binding site of each second anti-idiotypic antibody is made up of amino acids arranged so as to form a 3-dimensional mirror image of the original antigen of interest that was administered to the first host species. Both the original antigen of interest and the binding site of the second anti-idiotypic antibody “fit” into the antigen-combining site of the first antibody and can bind to and sequester the first antibody. Therefore, when a variety of purposefully designed anti-idiotypic antibodies are added to a biological sample containing many different antibodies to various antigens, any antibodies that recognize the binding site of an anti-idiotypic antibody are bound and sequestered and can be removed from the sample.

Since the anti-idiotypic reagents comprise regions or segments that are mirror images of antigens, they may also be used to elicit an immune response to those antigens, i.e. as vaccines. The mirror image binding sites of the anti-idiotypic reagents mimic the 3-dimensional structure of the antigens so that antibodies raised to the anti-idiotypic reagents in a third host, such as a human or a third animal species, also bind to authentic antigens when encountered, e.g. when a vaccinated subject is infected with an agent comprising the authentic antigens, antibodies acquired to the anti-idiotypic reagents recognize the authentic antigen and bind to it. However, the idiotypic reagents are advantageous because they are not infectious.

Methods of raising antibodies to antigens (both those present e.g. on an infectious agent or those which are present on other polypeptides, nucleic acids, polymers, saccharides, conjugated low molecular weight molecules, such as haptens, etc.) are known in the art. Briefly, molecular recognition of an antigen by the immune system results in selective production of antibodies that bind to the specific antigen. As used herein “antibody production” refers to the process of creating a usable specific antibody, and generally includes steps of immunogen preparation, immunization, hybridoma creation, collection, screening, isotyping, and (optionally) purification and labeling for direct use in a particular method.

Antibody production typically involves preparation of antigen samples and their repeated injection (boosters) into laboratory or farm animals to evoke high expression levels of antigen-specific antibodies in the serum, which can then be recovered from the animal. Polyclonal antibodies are recovered directly from serum (bleeds). Monoclonal antibodies are produced by fusing antibody-secreting spleen cells from immunized mice or rats with immortal myeloma cells to create monoclonal hybridoma cell lines that express the specific antibody in cell culture supernatant.

Planning and implementation may also include steps of: synthesizing and/or purifying the target antigen (e.g., peptide or hapten); choosing an appropriate immunogenic carrier protein; conjugating the antigen and carrier protein to create a higher immunogenicity by a weak immunogen; immunize animals using appropriate schedules and adjuvant formulae; and screening serum (or hybridoma) for antibody titer and isotype.

Antibody purification may include isolation of antibody from serum (polyclonal antibody), ascites fluid or culture supernatant of a hybridoma cell line (monoclonal antibody). Purification methods may be crude (one or more steps of precipitation of a subset of total serum proteins that includes immunoglobulins; general purification including one or more steps of affinity purification of certain antibody classes (e.g., IgG) without regard to antigen specificity, etc.) or highly specific, including one or more steps of affinity purification of only those antibodies in a sample that bind to a particular antigen molecule. For the present invention, antibodies are generally purified by a highly specific affinity purification of IgG that are specific to the antigen of interest.

Antibody characterization generally involves three kinds of activities that are performed at various stages throughout an entire antibody production and purification project: screening (identifying antibody samples having antigen-binding specificity), titering (measuring antibody concentration and functional assay titer) and isotyping (determining a monoclonal antibody class and subclass identity). Screening is generally required during production to identify which animals and hybridoma clones are producing a high level of antigen-specific antibody. This is usually accomplished using ELISA techniques. Antibody concentration can be estimated using either a general protein assay or a species- and immunoglobulin-specific method, such as with specialized microagglutination assay kits. Antibody titer is related to concentration but refers more specifically to the effective potency of a given antibody sample. Measuring titer usually means determining the functional dilution of an antibody sample necessary for detection in a given assay, such as an indirect ELISA.

Other steps of obtaining suitable antibodies may include:

Isotyping, which involves determining the class (e.g., IgG vs. IgM) and subclass (e.g., IgG1 vs. IgG2a) of a monoclonal antibody. This is a critical step in antibody production, as it is necessary for choosing an appropriate purification and modification method for the molecule. Isotyping is most easily accomplished with commercial, ready-to-use antibody isotyping kits.

Antibody fragmentation: purified antibodies can be modified for particular uses by several methods including fragmentation into smaller antigen-binding units, conjugation with enzyme or other detectable markers, and/or immobilization to solid supports. In the practice of the present invention, antibodies may be used in whole-molecule form. However, in other aspects, the reagent that is used is an antibody whose nonessential portions have been removed, but in which the antigen-binding portions or mirror-image binding sites are retained. For example, Fab and F(ab)′₂ are antibody fragments of IgG that may be created and utilized.

In some aspects, the usefulness of antibodies depends upon having a mechanism to secondarily detect the antibody. For example, antibodies may be labeled with a detectable label and/or mechanisms for attaching or immobilizing antibodies to chromatography media (e.g., beaded agarose resin), may be used. The invention also encompasses first antibodies and/or anti-idiotype polypeptides conjugated to a label capable of producing a detectable signal. Such labels are known in the art and include, but are not limited to, radioisotopes, enzymes, fluorescent compounds, chemiluminescent compounds, and bioluminescent compounds, and biotin-avidin labeling systems. The labels may be covalently linked to the polypeptide, or conjugated through a secondary reagent, such as a second antibody, protein A, or a biotin-avidin complex. Further information is found, for example, in: Alberts, B., et al. (1983), Molecular Biology of the Cell, Garland Publishing, Inc., New York, NY; Harlow, E. and Lane, D. (1988), Antibodies: A Laboratory Manual, Cold Spring Harbor Laboratory, Cold Spring Harbor, NY.; and Sites, D. P., et al. (1976), Basic & Clinical Immunology, Lange Medical Publication, Los Altos, CA; the contents of each of which is hereby incorporated by reference in entirety.

Such techniques are used to generate a first antibody to an antigen of interest and a second anti-idiotypic antibody to the paratope (antigen binding region) of the first antibody. The second antibody is an anti-idiotypic antibody with a “binding site” that mimics, both functionally and structurally, the antigen to which the first antibody was raised against.

An exemplary protocol is as follows:
1. An emulsion (e.g. an oil-in-water or water-in-oil emulsion) comprising an antigen of interest or agents comprising the antigen(s) of interest is formed. The colloidal setting of the Stator Gap ranges e.g. from 0.001-0.009 inches.
2. The emulsion is administered to goats or another suitable animal species. The administered volume of the emulsion typically ranges from about 1 to 5 ml per animal, and the interval between administrations typically varies from about 1 to 5 weeks. The collection of serum or plasma from the animal occurs after the 2nd to 10th injection of the emulsion.
3. Antibodies are recovered from the serum or plasma and Fab fragments are formed. Formation is, for example, by Papain enzymatic activity or by another suitable technology. Purification of the produced Fab is accomplished by any of various suitable techniques, e.g. a Fabsorbent™ F1 PHF, a synthetic-ligand light-chain binding adsorbent, Protein L, etc.
4. Pooled Fab is concentrated to a range between about 10 to 200 mg/ml, as an emulsion. The emulsification of the Fab preparations could use various oil phases, emulsifiers, and colloidal mill settings.
5. The Fab emulsions are administered preferably to laying chickens or another suitable animal species that is different than the species used in producing the Fab.
6. The volume, frequency, and interval of time between the injections of Fab to laying chickens (or to another animal species) could vary.
7. Eggs are collected from the birds effective 2 weeks post each of the administrations of the Fab emulsion. Collection extends until the end of the egg production cycle at the age of about 1.5 years of the birds. Molting of the chicken layers results in extension of the laying cycle, and resumption of administration of Fab emulsion in varying volume, frequency, and interval of time between boosters. This approach ensures the continuous acquisition of high concentrations antibodies that are specific to the idiotypes of the Fab emulsions.
8. Yolk of eggs collected from each group of birds is pooled, an equal volume of PBS buffer is added, the mixture is vortexed (e.g. for 5 minutes) and then centrifuged. The supernatant is collected from each pool, forming multiple pools of anti-idiotypes, each of which is specific to antibody Combining Sites (ACS), the idiotypes of the produced isotypes with a three-dimensional mirror image to the selected antigens of interest. The anti-idiotype antibodies may be cleaved e.g. by papain, to form Fab fragments.
9. Each pool corresponding to a single anti-idiotype, collected from eggs of 10 birds/group, is adjusted to a concentration of about 10 to 100 mg/ml. Confirmation of specificity of each chicken anti-idiotype to the Fab idiotypes is performed by titration of the chicken yolk supernatant against its eluted Fab, by procedure of Agar Precipitin Test, designating a titer by the maximum dilution of the chicken yolk supernatant that still forms a precipitin line against a Fab concentration of e.g. 10-100 mg/ml.
10. Equal volumes of each of the pooled anti-idiotypes are pooled together to form a reagent to eliminate cross-reacting antibodies from other commercial indirect ELISA. The anti-idiotype antibodies may be cleaved e.g. by papain, to form Fab fragments, and the Fab fragments are then used as the reagent.
11. This reagent is added in equal volume to any human fluid sample, left for about 15-60 minutes, followed by centrifugation to discard the precipitate.
12. The supernatant is then analyzed on commercial indirect ELISA, targeting the identification and/or quantification of isotypes specific to an agent of interest without any, or with only very low levels, of cross-reactivity, since potential cross-reacting antibodies will have been removed by the reagent in step 11.

It should be understood that the invention is not limited to specific sequences of any anti-idiotypic reagents/polypeptides disclosed herein as but encompasses anti-idiotypic reagents/polypeptides for all antigens of interest to which antibodies can be raised. Anti-idiotypic polypeptides having varying hyper-variability in their sequences at their kappa and lambda light chains, and in the variable regions of their heavy chains, are encompassed.

### ELISAs

Indirect enzyme-linked immunosorbent assays (ELISAs) are commonly used in analytical biochemistry assays, incorporating a solid-phase type of enzyme immunoassay (EIA) to detect and/or quantify the antibodies in a liquid sample by their binding to coated antigens on a solid matrix. If antibodies to the antigen are detected, this is evidence that the subject from whom the sample was obtained was previously exposed to/infected with, the antigen or an agent that bears the antigen.

Unfortunately, cross-reactivity between antigens is frequently a problem. Cross-reactivity occurs when e.g. when two (or more) antigens (e.g. for different variants or strains, or even species, of an infectious agent), only one of which is an antigen of interest, have similar structural regions so that antibodies generated to one of the antigens also recognize the others. Cross-reactivity of the antibodies then results in false positives when an indirect ELISA test is conducted on a sample from the subject, and subjects may be diagnosed as having or having had an infection caused by one infectious agent when in fact, this is not true. Rather, the subject has or had an infection caused by a different infectious agent whose antibodies cross-react. This makes it extremely difficult to correctly diagnose, control by specific vaccines, and to treat illnesses by therapeutics, and to compile statistics concerning the origin and the epizooteology of an infectious agent. In addition, this cross-reactivity will affect the interpretation of the titers that are supposed to be specific to antigen of interest, such as vaccine titers, making it difficult to judge the efficacy of a vaccine, and to schedule the time for a booster, as currently witnessed in the hardship of determining the appropriate time for administering a third dose of the Covid 19 vaccine.

The present invention provides improved indirect ELISA protocols in which samples to be tested are first pretreated with at least one anti-idiotypic polypeptide as described herein. Generally, a plurality of anti-idiotypic polypeptides is used, each of which comprises a mirror image of an antigen (antigenic determinant, epitope, etc.) that is not the antigen of interest but is closely related thereto structurally. Antibodies present in the sample bind to and are captured by the anti-idiotypic polypeptides and are removed from the sample. The antibodies that are left behind in the sample can then be detected using a routine indirect ELISA assay in which the captured molecule is an antibody that is highly specific to the antigen of interest.

In some aspects, the agent of interest is SARS-COV-2. Methods of neutralization of cross-reacting antibodies that are specific to antigens of HCoVs (229E, NL63, OC43, and HKU1) and to antigens of zoonotic SARS-COV-1 and MERS-COV, are provided. Neutralization occurs during pre-treatment of a human fluid sample by the anti-idiotypic reagents, before the human fluid sample is analyzed using a commercial indirect ELISA system for capturing antibodies specific to SARS-COV-2.

In some aspects, the step of pretreating ELISA samples increases the mean % difference between a sample positive for an antibody of interest and a sample that is negative for the antibody of interest by at least 90%, such as about 90, 91, 92, 93, 94, 95, 96, 97, 98 or 99%. For example, the mean % difference between a SARS-COV-2 PCR-positive and SARS-COV-2 PCR-negative sample is generally more than 90% and typically more than 95%.

Accordingly, this disclosure provides methods of conducting indirect ELISA assays designed to detect antibodies to an infectious agent or any other molecule that can induce immunity in any host species. The methods comprise obtaining a biological sample from a subject. The biological sample may be, for example, blood, plasma, saliva, mucous, and any extracted antibody from the various tissues of different hosts, etc.

The methods also include a step of contacting the biological sample with a composition comprising at least one anti-idiotypic polypeptide that is specific for a variable region of an antibody combining site (ACS) of an antibody that specifically cross-reacts with at least one antigen of the infectious agent, or any molecule that can induce an immune response, when free or conjugated to a carrier. The step of contacting causes the at least one anti-idiotypic polypeptide to bind to the cross-reactive antibody and form an anti-idiotypic polypeptide: antibody complex. Those of skill in the art are familiar with containers that are suitable for conducting ELISAs and related biochemical assays, e.g. multi-wells microtiter plates, test tubes, multi-channel pipettes, incubators, ELISA reader, etc.

Another step of the methods is removing the anti-idiotypic polypeptide: antibody complex from the sample, i.e. the complex formed by the anti-idiotypic polypeptide and the cross-reacting antibody is removed so that the cross-reacting antibody will not interfere with the subsequent indirect ELISA assay. In order to remove the complex, the anti-idiotypic polypeptide(s) may be attached to a solid support, such as beads (e.g. magnetic beads), the walls of a container, etc. Alternatively, the complex may form a precipitate that is then removed. Removal is performed e.g. by centrifugation, filtration, etc.

The user is then free to conduct the indirect ELISA assay for antibodies to the infectious agent, or to any other antigens, such as vitamins, hormones, or any metabolite, or molecule, even to very small molecules such as haptens, with a low level of or interference from cross-reacting antibodies. Cross-reacting antibodies have been removed fully or at least to a large extent (e.g. at least 90, 91, 92, 93, 94, 95, 96, 97, 98 or 99% are removed).

### Vaccines

Vaccine preparations by the same ‘One Process’ comprising one or more anti-idiotypic polypeptides, as described herein, are also encompassed by the invention. In the discussion below, SARS-Cov-2 is described as an exemplary prototype.

The current developed vaccines for COVID 19 that have been given ‘Emergency Licensing’ are reported to result in vaccine reactions, such as fever, chills, headache, muscular and joint ache, fatigue, and loss of appetite for a period of around 48 hrs post vaccination, with a higher frequency of reaction after the second dose (CDC, 2021). In addition, these vaccines induce immunity against only one protein namely, the spike protein, in which mutations in its gene cause mutants to have significantly lower neutralization by vaccine-induced antibodies (Liu et al, 2021). The current vaccines ignored the role of the lipid bilayer that covers the virus (Abou-Farha et al, 2020), knowing that lipids have very weak immunogenicity.

In contrast, the mirror image, anti-idiotype reagents, disclosed herein, do not have these drawbacks. The disclosed anti-idiotype reagents are based on the whole outer three-dimensional structure of the virus, including the low immunogenic lipids and the glycogen portion of the spikes present on the coronavirus. The anti-idiotype reagents comprise mirror images of amino acid chains of idiotypes that are highly immunogenic, giving the anti-idiotype vaccines against SARS-COV-2, or against any other type of CoVs, a significant advantage over the current ‘Emergency Licensed’ vaccines. The anti-idiotype vaccines induce polyvalent antibodies to many antigenic determinants on an infectious agent such as SARS-COV-2, including the weak lipid and glycogen antigenic determinants, making it difficult for a mutant to escape from the polyvalent antibodies acquired as a result of administration of an anti-idiotype vaccine.

A “vaccine” composition as used herein refers to a composition comprising at least one anti-idiotype polypeptide as described herein and a pharmaceutically acceptable carrier, solvent, excipient and/or an adjuvant. As used herein, “pharmaceutical composition” refers to a composition suitable for administration to a subject animal, including humans. In the present context, a pharmaceutical composition comprises a pharmacologically effective amount of anti-idiotype polypeptide molecule and a pharmaceutically acceptable carrier, solvent or excipient. Accordingly, pharmaceutical compositions of the present invention encompass any composition made by admixing at least one anti-idiotype polypeptide in accordance with the present invention and a pharmaceutically acceptable carrier.

A subject to whom the vaccine is administered may be a mammal, such as a human. However, veterinary applications of this technology are also encompassed so that companion pets (e.g. dogs, cats, ferrets, etc.); and/or working animals (cows, horses, camels, donkeys, etc.); and/or animals in preserves, laboratories or zoos, etc.; and even non-mammals such as poultry, pet birds, amphibians, reptiles, fish, etc.; can be immunized using the safe anti-idiotype vaccines prepared by the described ‘One Process’.

A “pharmaceutically acceptable carrier” refers to a clinically useful solvent, dispersion medium, coating, isotonic and absorption delaying agent, buffer, and excipient, such as a phosphate buffered saline solution (PBS), aqueous solutions of dextrose or mannitol, and emulsions, such as an oil-in-water or water-in-oil emulsions, and various types of wetting agents, immunostimulators, and/or adjuvants. Suitable pharmaceutical carriers and formulations are described in Remington's Pharmaceutical Sciences, 21st Ed. (Mack Publishing Co., Easton, 2006). Pharmaceutical carriers useful for the composition depend upon the intended mode of administration of the active agent. Typical modes of administration include but are not limited to: parenteral administration, including subcutaneous, intramuscular, intravenous or intraperitoneal injection; transdermal and transmucosal administration; inhalation; etc.

The pharmaceutical compositions may optionally include an adjuvant. An adjuvant is a substance which enhances the body's immune response to an antigen. Many are known in the art and can be utilized in the vaccine compositions of the present invention, including but not limited to: alum (aluminum phosphate, aluminum hydroxide, aluminum potassium sulfate), a cross-linked polyacrylic acid polymer, dimethyldioctadecylammonium bromide (DDA), lactoferrin, an IFN-gamma derivative, a non-ionic detergent, a vegetable oil, surface active substances (including lysolecithin, pluronic polyols, polyanions), various peptides, oil or hydrocarbon emulsions (e.g. oil-in-water emulsion), keyhole limpet hemocyanins, squalene based adjuvants (such as MF59), montanide, RIM adjuvant, complete Freud's adjuvant and incomplete Freud's adjuvant, MPL, muramyl dipeptide, TLR ligand based adjuvants, CpG oligonucleotides, non-CpG oligonucleotides, saponins such as QS-1, ISCOM, ISCOMATRIX, vitamins, and immunomodulants such as cytokines, and the like. Also includes is the saponin adjuvant described in published U.S. patent application No. 20210228709; rBCG as described in published U.S. patent application No. 20210222179; Dectin-2 ligand vaccine adjuvant described in published U.S. patent application No. 20210205445; and vaccine adjuvants described in published U.S. patent application No. 20210187104. The entire contents of all published US patent applications listed herein are hereby incorporated by reference in entirety.

The vaccine compositions described herein are used prophylactically and/or therapeutically. A “prophylactic” treatment is a treatment administered to a subject who does not exhibit signs of an infection for the purpose of reducing the likelihood of an infection, decreasing the risk of developing pathology from the infection, decreasing the severity the infection, decreasing the duration of an infection should one occur, and/or decreasing the risk of transmitting the infection to another subject. A “therapeutic” treatment is a treatment administered to a subject who exhibits signs or symptoms of infection for the purpose of reducing the severity of infection, shortening the duration of infection, reducing or eliminating signs or symptoms of an infection, reducing viral shedding of the infection, and/or reducing the likelihood of transmitting the infection.

In addition, the vaccines may be a therapeutic cocktail of anti-idiotype antibodies. The ‘One Process’ method produces anti-idiotype antibodies that are specific to epitopes on harmful antigens that cause injuries in humans and in different animal species, e.g. for their inclusion in vaccines to produce therapeutics for reacting against the paratope of antibodies that are involved in autoimmune diseases, due to their binding to self-antigens in the body of humans and animals.

### Agents for Development of Anti-Idiotypes

Anti-idiotype reagents are developed, as described herein, for a wide variety of agents comprising mirror images of antigens that are suitable for this purpose. The agents comprise at least one antigen to which an antibody can be raised to its epitope; the paratope of the antibody that is raised then serving as the “antigen” or antigenic region in another host species to which the anti-idiotype antibodies are in turn raised specifically to such a patratope. In other words, the agents comprise at least one antigen of interest that is suitable for creation of at least one first antibody and at least one second antibody to the paratope of the at least one first antibody, the at least one second antibody containing amino acid residues that form a mirror image of the at least one antigen of interest. Thus, the antigens are generally antigens for which at least one antibody is raised in first chosen animal species, e.g. a first antibody, and then the first antibody serves as the template for raising a second antibody, in another chosen animal species, in which this second antibody has a paratope that is specific to the variable regions of the first antibody. The second antibody is an idiotypic, mirror image of the original antigen (or antigenic region or epitope). Most antigens are macromolecules and include, for example proteins, polysaccharides, lipids, DNA, RNA, etc., and combinations of these.

In some aspects, the antigens are associated with agents that cause disease, such as infections agents, and include, for example, viruses, bacteria, protozoa, fungi, or they could be allergens, toxins, and other injurious immunogenic molecules that are greater in molecular weight than haptens, or they could be haptens conjugated to a carrier, enabling them to be immunogenic. However, the current methodology is not limited to infectious agents but applies to any molecule to which a first (primary) antibody can be raised, so that the first antibody then serves as a template for production of a second anti-idiotypic antibody to the paratope of the first antibody. Any antigen which is typically detected by an ELISA test and/or for which it is desirable to have a vaccine available, can be an antigen for which an anti-idiotypic reagent is developed, including, microbes, allergens, nutrients, hormones, neurotransmitters, cytokines, immunomodulators, toxins, metabolites, etc.

In some aspects, the agent with at least one antigen that is suitable for creation of a mirror image anti-idiotype as described herein is a virus, examples of which include but are not limited to: a coronavirus, and any animal and human viruses that are involved in subclinical and clinical diseases. Example, Hepatitis E, HIV, Flavi virus, Avian Influenza, Ebola, Hantavirus, Monkey Pox, Nipah virus, West Nile Fever, etc.

In some aspects, the infectious agent is a coronavirus. The coronavirus may be any of the four genera: Alphacoronaviruses and betacoronaviruses (which infect mammals) or gammacoronaviruses and deltacoronaviruses which primarily infect birds. The genus Alphacoronavirus includes species: Alphacoronavirus 1 (TGEV, Feline coronavirus, Canine coronavirus), Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2 and Scotophilus bat coronavirus 512. The genus Betacoronavirus include the species: Betacoronavirus (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-COV, SARS-COV-2 and variants thereof) and Tylonycteris bat coronavirus HKU4. The genus Gammacoronavirus includes the species: Avian coronavirus and Beluga whale coronavirus SW1. The genus Deltacoronavirus includes the species: Bulbul coronavirus HKU11 and Porcine coronavirus HKU15.

In some aspects, the agent with at least one antigen that is suitable for creation of a mirror image anti-idiotype as described herein is a bacterium, examples of which include but are not limited to opportunistic bacteria, carrier bacteria, and bacteria that cause primary and secondary infections in animals and humans. Examples are Salmonella organisms, Campylobacter, Brucella, Neisseria, Mycobacteria, Nocardia. Listeria, Francisella, Legionella, Yersinia, Chlamydia, etc.

In some aspects, the agent comprising at least one antigen that is suitable for creation of a mirror image anti-idiotype as described herein is a protozoa, examples of which include but are not limited to: Toxoplasma, Giardia, Entamoeba histolytica, Trypanosoma spp., etc.

In some aspects, the agent comprising at least one antigen that is suitable for creation of a mirror image anti-idiotype as described herein is a fungus, examples of which include but are not limited to: Aspergillus, Candidia, Cryptococcus, Histoplasma, Blastomyces, Pythium insidiosum, Lagenedium spp., Rhinosporidium seeberi, etc.

In some aspects, the agent comprising at least one antigen that is suitable for creation of a mirror image anti-idiotype as described herein is an allergen, examples of which include but are not limited to: drug allergens, food allergens, insect allergens, latex allergen, mold allergen, pet allergen, pollen allergen, etc.

In some aspects, the agent comprising at least one antigen that is suitable for creation of a mirror image anti-idiotype as described herein are hormones, examples of which include but are not limited to: T3, T4, Melatonin, Progesterone, Testosterone, Cortisol, Insulin, Estrogen, etc.

In some aspects, the agent comprising at least one antigen that is suitable for creation of a mirror image anti-idiotype as described herein are neurotransmitters, examples of which include but are not limited to: Amino acids such as gamma-Aminobutyric Acid (GABA), glutamate, aspartate, D-serine; Gasotransmitters such as NO, CO, H2S; Monoamines such as dopamine, norepinephrine (noradrenaline), epinephrine (adrenaline), histamine, serotonin (SER, 5-HT); other hormones.

In some aspects, the agent comprising at least one antigen that is suitable for creation of a mirror image anti-idiotype as described herein are cytokines, examples of which include but are not limited to: IL-1, IL-6, IL-10, Interferons, Tumor Necrosis Factor (TNF), TGF-Beta, etc.

In some aspects, the agent comprising at least one antigen that is suitable for creation of a mirror image anti-idiotype as described herein are immune modulators, examples of which include but are not limited to: Thalidomide, lenalidomide, pomalidomide, Bacillus Calmette-Guerin, Imiquimod, etc.

In some aspects, the agent comprising at least one antigen that is suitable for creation of a mirror image anti-idiotype as described herein are toxins, examples of which include but are not limited to: Living organisms produced toxins, such as those produced by microbes, plants, fungus, or venoms produced by animals; Physical toxicants such as coal dust, asbestos, silicon dioxide, asphyxiant gases, etc.

In some aspects, the agent comprising at least one antigen that is suitable for creation of a mirror image anti-idiotype as described herein is a nutrient, examples of which include but are not limited to: Vit A, C, D, E, K, B (thiamine, riboflavin, niacin, pantothenic acid, biotin, B6, B12m and folate), proteins and related peptides, lipopolysaccharides; carbohydrates, lipids, etc.

In some aspects, the agent comprising at least one antigen that is suitable for creation of a mirror image anti-idiotype as described herein is a metabolite, examples of which include but are not limited to: metabolites involved in survival of animals, humans and plants (normal growth, development, and reproduction) such as polyols, organic acids, nucleotides; other metabolites such as the serum metabolome including members of Acyl glycines, Acyl phosphates, Alcohol phosphates, aldehydes, alcohols etc.

The agent comprising at least one antigen that is suitable for creation of a mirror image anti-idiotype can be a microbe or a molecule involved in human diseases and veterinary diseases of different domestic and wild animal species. Also includes are viruses, bacteria, protozoa, fungi, toxins, allergens, tumor antigens, and specific harmful idiotypes in autoimmunity diseases of humans and animals. Therapeutics produced in the ‘One Process’ may be active against paratopes of antibodies causing autoimmune diseases including but not limited to the following harmful antibodies: anti-actin, anti-centromere, anti-ganglioside, anti-mitochondria, anti-neutrophils, anti-nuclear, anti-signal recognition peptide, anti-smooth muscle, anti-glomerular basement membrane, anti-parietal cell, anti-liver-kidney microsomes, anti-soluble liver antigen, etc.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Example 1

The problem of cross-reactivity, which leads to false positive results, is prevalent in the domain of analysis using commercial indirect ELISA kits. Cross-reactivity in ELISAs lowers the reliability of the diagnostic objective and vitiates its benefits in control programs.

SARS-COV-2 was selected as an exemplary model to develop the present ‘One Process’ invention. However, the methodology is applicable to other targets of interest namely, the applicability of the ‘One Process’ to produce anti-idiotypes for elimination of cross reactivity in indirect ELISA systems, and for inclusion of the same prepared anti-idiotypes in vaccines.

Coronaviruses (CoVs) are enveloped positive-sense RNA viruses that belong to the Coronaviridae family, phylogenetically subdivided into the α, β, γ, and δ genera. The β-coronaviruses include SARS-COV, MERS-COV, and SARS-COV-2, viruses that are identified as the causative agents of zoonotic infections, with many overlapping symptoms. Severe Acute Respiratory Syndrome (SARS-COV) emerged in Southern China in 2003, while Middle East Respiratory Syndrome (MERS-COV) appeared in Saudi Arabia almost a decade after the SARS-COV outbreak. The most recent pandemic, caused by Severe Acute Respiratory Syndrome Coronavirus 2 (SARS-COV-2), started in the Chinese province of Guangdong in November 2019.

To practice the ‘One Process’ method and obtain anti-idiotypic polypeptides for a cross-reacting antigen, it is necessary to identity the cross-reacting antigen i.e. a problematic, cross-reacting antigen, antibodies to which interfere with or are likely to interfere with an indirect ELISA test to detect antibodies to an antigen of interest. For the present ‘One Process’ method, generally a plurality of cross-reacting antigens are identified. Once identified, it is necessary to obtain a quantity of the cross-reacting antigen(s) sufficient to obtain the first antibodies thereto and then prepare the anti-idiotypic polypeptides to those first antibodies. The method of propagating a cross-reacting antigen is dependent on its nature. For example, a cross-reacting virus is propagated in a suitable cell culture system; bacteria and fungi are propagated in suitable bacteriological media; and a parasite is propagated in an in vitro system or in animals. In some cases, other antigenic molecules or portions of molecules are recovered from environmental niches or from animals or humans. Some of these cross-reacting molecules could be non-immunogenic haptens, with low molecular weight, however, when when conjugated to a carrier, it enables the hapten to become an imunogen. In the ‘One Process’ method, once produced, the cross-reactive antigens are produced (propagated), they are concentrated, and the concentration is adjusted as needed to perform subsequent steps of the method. If the propagated antigen is present on a live microorganism, then the inactivation of the propagated microorganism is by formalin or another suitable inactivating substance.

An exemplary protocol for preparation of anti-idiotypes for coronaviruses namely to cross-reacting types of 229E. NL63, OC43, HKU1, SARS-COV-1, MERS-COV, and SARS-CoV-2 is as follows:
1. Formation of an emulsion for each of the above seven coronaviruses types (e.g. an oil-in-water or water-in-oil emulsion) comprising the inactivated propagated infectious agents. The colloidal setting of the Stator Gap ranges from 0.001-0.009 inches, producing appropriate micelle sizes in the prepared emulsions.
2. The emulsion is administered to goats or another suitable animal species. The administered volume of the emulsion typically ranges from about 1 to 5 ml per animal, and the interval between administrations typically varies from about 1 to 5 weeks. The collection of serum or plasma from the animal occurs after the 2nd to 10th injection of the emulsion.
3. Antibodies are recovered from the serum or plasma and Fab fragments are formed. Formation is, for example, by Papain enzymatic activity or by another suitable technology. Purification of the produced Fab is accomplished by any of various suitable techniques, e.g. a Fabsorbent™ F1 PHF, a synthetic-ligand light-chain binding adsorbent, Protein L, etc.
4. Pooled Fab is concentrated to a range between about 10 to 200 mg/ml, as an emulsion. The emulsification of the Fab preparations could use various oil phases, emulsifiers, and colloidal mill settings.
5. The Fab emulsions for one idiotype are administered to e.g. laying chickens or another suitable animal species that is different than the species used in producing the Fab.
6. The volume, frequency, and interval of time between the injections of Fab to laying chickens (or to another animal species) could vary.
7. Eggs are collected from the birds effective 2 weeks post each of the administrations of the Fab emulsion. Collection extends until the end of the egg production cycle at the age of about 1.5 years of the birds. Chicken layers could be molted to extend their laying cycles beyond the 1.5 years of age, while boosting their immunity by additional administration of the Fab emulsions. This approach ensures the continuous acquisition of high concentrations of antibodies that are specific to the idiotypes of the Fab emulsions.
8. Yolk of eggs collected from each group of birds is pooled, an equal volume of PBS buffer is added, the mixture is vortexed (e.g. for 5 minutes) and then centrifuged. The supernatant is collected from each pool, forming multiple pools of anti-idiotypes, each of which is specific to antibody Combining Sites (ACS) containing idiotypes with specificity to antigens carried by coronaviruses of the type 229E, NL63, OC43, HKU1, SARS-COV-1, MERS-COV, and SARS-COV-2.
9. Each pool corresponding to a single anti-idiotype, collected from eggs of 10 birds/group, is adjusted to a concentration of about 10 to 100 mg/ml and labelled respectively as, chicken anti-idiotype 229E, chicken anti-idiotype NL63, chicken anti-idiotype OC43, chicken anti-idiotype HKU1, chicken anti-idiotype SARS-COV-1, chicken anti-idiotype MERS-COV, and chicken anti-idiotype SARS-COV-2. The confirmation of specificity of each chicken anti-idiotype to the Fab idiotypes is performed by titration of the chicken yolk supernatant against eluted Fab, by procedure of Agar Precipitin Test, designating a titer by the maximum dilution of the chicken yolk supernatant that still forms a precipitin line against a Fab concentration of e.g. 10-100 mg/ml.
10. Equal volumes of each of the anti-idiotype pools (chicken anti-idiotype 229E, chicken anti-idiotype NL63, chicken anti-idiotype OC43, chicken anti-idiotype HKU1, chicken anti-idiotype SARS-COV-1, chicken anti-idiotype MERS-COV) are pooled together to form a multi-anti-idiotypic reagent to eliminate cross-reacting antibodies from other commercial indirect ELISA systems. The novel product is labelled e.g. ‘Eliminator of cross-reacting isotypes to SARS-COV-2’, abbreviated to ‘ECI-SARS-COV-2’. This labelling is changed according to modification of the above protocol to eliminate cross-reacting antibodies from other commercial indirect ELISAs with a new targeted isotype (X), thus the abbreviated name will be changed to ‘ECI-X’.
11. This reagent is added in equal volume to any human fluid sample, left for about 15-60 minutes, followed by centrifugation to discard the precipitate, and the supernatant is then analyzed on commercial indirect ELISA, targeting the identification or quantification of antibodies specific to an agent of interest.

### Vaccines

The anti-idiotypes acquired from chicken yolks, or in sera or plasma of other animal species, are each used as a vaccine after going through the following steps:
1. Each acquired anti-idiotype, harvested from chicken or other animal species in the above, namely, the anti-idiotypes to 229E, NL63, OC43, HKU1, SARS-COV-1, MERS-COV, and SARS-COV-2, is subjected to treatment with a papain enzyme to form Fab fragments. The Fab fragments are purified e.g. by Fabsorbent™ F1 PHF, and the pure Fab is eluted at a pH of 4.0.
2. The seven pools of Fab are labelled Fab anti-idiotype 229E, Fab anti-idiotype NL63, Fab anti-idiotype OC43, Fab anti-idiotype HKU1, Fab anti-idiotype SARS-COV-1, Fab anti-idiotype MERS-COV, and Fab anti-idiotype SARS-COV-2.
3. Each of the Fab idiotype preparations is adjuvanted with e.g. aluminum salt, a safe approved adjuvant, revised by FDA in 2020, or another adjuvant approved by FDA for use in animals and humans vaccines. The adjuvanted Fabs serve as an anti-idiotype vaccine for a specific type of infectious agent, such as a CoV type. These adjuvanted products are labelled: Anti-idiotype Vaccine 229E, Anti-idiotype Vaccine NL63, Anti-idiotype Vaccine OC43, Anti-idiotype Vaccine HKU1, Anti-idiotype Vaccine SARS-COV-1, Anti-idiotype Vaccine MERS-COV, and Anti-idiotype Vaccine SARS-COV-2. In addition, these prepared vaccines could incorporate more than one anti-idiotype, resulting in bivalent, trivalent, or even polyvalent vaccines.

This ‘One Process’ can be modified by selecting different idiotypes of interest to target idiotypes with other specificities for different commercial indirect ELISAs or different vaccines. The rest of the process remains the same to end in production of anti-idiotype reagents to neutralize the cross-reacting isotypes, and production of the anti-idiotype vaccines to protect against an antigen of interest, or against more than one antigen.

### Example 2. Elimination of Cross-Reactivity by the Invented Anti-Idiotypic Product

The aim of this Example is to show the advantage of treating the human samples with the invented product to attain wider significant differences between negative and positive samples, using the SARS-COV-2 model.

A commercial indirect ELISA to detect antibodies to recombinant N Protein of SARS-CoV-2 was tested and was able to reach to a maximum mean % difference between negative and positive samples equivalent to only 36%, and it enabled to uncover the percentage of false IgG positives of only 40%.

Accordingly, we attempted the elimination of cross-reactivity by increasing the mean % difference between the negative and positive serum samples, as follows:

Serum samples were divided into two portions of equal volume (0.5 ml) each, in which one portion is treated with equal volume of the Anti-idiotypic Product produced as described in Example 1. The anti-idiotypic product (reagent) comprised Fab portions of anti-idiotypic antibodies against 229E. NL63, OC43, HKU1, SARS-COV-1, and MERS-COV. Two portions of each sample were analyzed in two trials, using different indirect ELISA systems for SARS-COV-2 for each trial. Each trial analyzed 20 positive human samples (positive by PCR for SARS-COV-2) and 20 negative samples (negative by PCR for SARS-CoV-2, and positive by PCR for one of the HCoVs types. More specifically, The SARS-CoV-2 PCR-negative samples in Trial 1 were positive for MERS-COV, while the SARS-CoV-2 PCR-negative samples in Trial 2 were positive for SARS-COV-1. The mean % difference between the SARS-COV-2 PCR-positives and PCR-negatives are shown in Table 1.

TABLE 1
Mean % difference by pre-treatment of human samples with invented
anti-idiotypic product before analysis on indirect ELISA

| | | Mean % difference between |
|---|---|---|
| | | SARS-CoV-2 PCR-positive |
| Trial | Application of | and SARS-CoV-2 |
| Number¹ | Invented Product | PCR-negative samples |
| 1 | + | 97.0^{a} |
| | − | 52.0^{b} |
| 2 | + | 94.0^{a} |
| | − | 56.0^{b} |

¹The SARS-CoV-2 PCR-negative samples in Trial 1 were positive for MERS-CoV. The SARS-CoV-2 PCR-negative samples in Trial 2 were positive for SARS-CoV-1.
^{a,b}The presence of different alphabetical superscripts following the means in the last column is indicative of the advantage of eliminating the cross-reactivity from the negative samples by the addition of the application of invented product that widened the difference between the PCR positive and negative samples for SARS-CoV-2, at a statistical difference of P < 0.01.

¹The SARS-COV-2 PCR-negative samples in Trial 1 were positive for MERS-COV. The SARS-COV-2 PCR-negative samples in Trial 2 were positive for SARS-COV-1. ^{a,b}The presence of different alphabetical superscripts following the means in the last column is indicative of the advantage of eliminating the cross-reactivity from the negative samples by the addition of the application of invented product that widened the difference between the PCR positive and negative samples for SARS-COV-2, at a statistical difference of P<0.01.

As can be seen, the step of pretreating the samples increased the mean % difference between SARS-COV-2 PCR-positive and SARS-COV-2 PCR-negative samples to over 90%.

### Example 3. Verification of Produced Anti-Idiotypes as CoV Vaccines

Data presented in this Example verified the mirror imaging of produced anti-idiotypes to their respective CoV types. Serial dilutions of seroconverted antibodies from individuals who were PCR positive to each respective anti-idiotype were reacted on Noble Agar Gel. The results of formation of precipitin lines, an indication of specificity of the acquired antibody to the antigen, are shown in Table 2.

TABLE 2
Specificity of invented anti-idiotypes to seroconverted antibodies
in humans infected with different CoVs and confirmed by PCR

Seroconverted

| Antibodies | No. | Mean Titer¹ to different invented anti-idiotypes | | | | | |
|---|---|---|---|---|---|---|---|
| to Different | sam- | | | | | SARS- | MERS- |
| CoV types | ples | 229E | NL63 | OC43 | HK 41 | CoV-1 | CoV |
| 229E | 5 | 1:120 | NA² | NA | NA | NA | NA |
| NL63 | 4 | NA | 1:180 | NA | NA | NA | NA |
| OC43 | 7 | NA | NA | 1:80 | NA | NA | NA |
| HKU1 | 7 | NA | NA | NA | 1:140 | NA | NA |
| SARS-CoV-1 | 10 | NA | NA | NA | NA | 1:160 | NA |
| MERS-CoV | 8 | NA | NA | NA | NA | NA | 1:240 |

¹Titer is the maximum dilution of the serum that still has enough antibodies to form a precipitin line with the invented anti-idiotypes.
²NA is a denotation of Not Applicable.

As can be seen in Table 2, the PCR positive test to each CoV did agree with the result of acquiring of antibodies that were highly specific to its respective anti-idiotype, confirming the mirror image nature of the anti-idiotype to its respective CoV epitopes. This shows the potential of these anti-idiotypes as components of vaccines, replacing the need for incorporating the CoV itself, and by that eliminating the hazard of failure in the inactivation process of the virus itself.

### Example 4. Applicability of this ‘One Process’ for Production of Covid-19 Vaccine

This same ‘One Process’ of forming anti-idiotype vaccines to other CoV types is also applicable to production of SARS-COV-2 anti-idiotypic vaccines. This is done by starting the first Step of the ‘One Process’ with each of four individual variants of SARS-COV-2 viral propagation in cell culture, and the following steps of the process stay the same (see Example 1). The produced anti-idiotype of each of the four variants, mirror images of respective SARS-CoV-2 viral variants, was verified by reaction of each of the ‘Anti-idiotype of SARS-COV-2 variant’ product on Noble Agar with a serial dilution of seroconverted antibodies from individuals who were PCR positive to different variants of COVID-19. The titer of each sample was deduced from formation of precipitin line by the highest dilution of the human serum sample. The mean titers are shown in Table 3.

TABLE 3
Verification of applicability of this Process
to Production of Covid-19 vaccine

| | | Mean sera titer specific to anti-idiotypes | | | |
|---|---|---|---|---|---|
| | | of initial and to that of the other three | | | |
| | | variants of SARS-CoV-2 | | | |
| Seroconverted | | | Great | South | |
| Antibodies to | | | Britain | African | India Delta |
| Four variants of | No. | Initial | Variant | Variant | Variant |
| SARS-CoV-2 | samples | strain | (B.11.7) | (B.1.351) | (B.1.617.2) |
| Initial Covid-19 | 20 | 1:640 | 1:320 | 1:80 | 1:80 |
| Strain | | | | | |
| (SARS-CoV-2) | | | | | |
| Great Britain | 15 | 1:640 | 1:1280 | 1:640 | 1:640 |
| Variant | | | | | |
| (SARS-CoV-2 VOC | | | | | |
| 202012/01, or | | | | | |
| B.1.1.7) | | | | | |
| South African | 12 | 1:160 | 1:80 | 1:640 | 1:80 |
| Variant | | | | | |
| (SARS-CoV-2 | | | | | |
| 20H/501Y.V2, or | | | | | |
| B.1.351) | | | | | |
| India Delta Variant | 10 | 1:80 | 1:640 | 1:80 | 1:1280 |
| (SARS-CoV-2 | | | | | |
| B.1.617.2) | | | | | |

All seroconverted samples from humans that were PCR positive to one of the four variants reacted to a different extent against the anti-idiotypes of the initial SARS-COV-2 isolate and to the three other variants of SARS-COV-2. It is apparent that the titers were higher when the reactions of acquired antibodies were directed against the anti-idiotype of the variant that caused the infection. This indicates that anti-idiotype vaccine production should include, in its first step of the process, the anti-idiotype of any emerging new variant(s), even before it or they become(s) globally predominant. Inclusion of anti-idiotype of new variants in production is implemented in the protocol of manufacturers, when the need arises, to change the vaccine for COVID-19 according to prevalence of the variant.

While the invention of the ‘One Process’ has been described in terms of its several exemplary embodiments, those skilled in the art will recognize that the invention can be practiced with modification within the spirit and scope of the appended claims. Accordingly, the present invention should not be limited to the embodiments, as described above, but should further include all modifications and equivalents thereof within the spirit and scope of the description provided herein.

We claim:

1. A method for conducting an indirect ELISA assay to detect antibodies to one or more epitopes of one or more antigens of an antigenic agent, comprising,
contacting a biological sample with a composition comprising at least one anti-idiotypic polypeptide that binds to a variable region of an antibody combining site (ACS) of an antibody that cross-reacts with the one or more epitopes of the one or more antigens, wherein the step of contacting causes the at least one anti-idiotypic polypeptide to bind to the antibody and form an anti-idiotypic polypeptide-antibody complex; then
removing the anti-idiotypic polypeptide-antibody complex from the sample; and then
conducting the indirect ELISA assay.

2. The method of claim 1, wherein the antigenic agent is an infectious agent.

3. The method of claim 1, wherein the at least one anti-idiotypic polypeptide is a mirror image of an epitope or an antigen of the infectious agent.

4. The method of claim 1, wherein the at least one anti-idiotypic polypeptide is immobilized on a support.

5. The method of claim 4, wherein the support is a bead.

6. The method of claim 1, wherein the at least one anti-idiotypic polypeptide is reacted with the biological sample in a liquid phase without immobilization of the anti-idiotypic polypeptide on a solid support, resulting in a precipitate that is removed by centrifugation.

7. The method of claim 1, wherein the one or more antigens is/are from a human, a non-human animal, a plant, or an environmental sample.

8. The method of claim 1, wherein the one or more antigens is/are present on a virus, bacteria, protozoa, fungus, allergen, toxin, or a free or conjugated molecule.

9. The method of claim 8, wherein the virus is a coronavirus.

10. The method of claim 9, wherein the antibody specifically cross-reacts with at least one coronavirus selected from the group consisting of HCoV-HKU1, HCOV-229E, HCoV-NL63, HCoV-OC43, SARS-COV-1, and MERS-COV and the indirect ELISA detects antibodies to SARS-Cov-2 antigen(s).

11. The method of claim 10, wherein the indirect ELISA detects the antibodies to SARS-Cov-2 antigen(s) qualitatively and/or quantitatively.

12. A composition comprising at least one anti-idiotypic polypeptide which is specific to a variable region of an antibody combining site (ACS) of one or more antibody isotypes that cross-react with coronavirus epitope(s).

13. The method of claim 12, wherein the at least one anti-idiotypic polypeptide is a mirror image of a coronavirus epitope.

14. The composition of claim 12, wherein the coronavirus is SARS-COV-2.

15. The composition of claim 12, wherein the coronavirus is HCoV-HKU1, HCOV-229E, HCoV-NL63, HCoV-OC43, SARS-COV-1 or MERS-COV.

16. A method of eliciting an immune response against a coronavirus in a subject in need thereof, comprising
administering to the subject a composition comprising
a therapeutically effective amount of at least one anti-idiotypic polypeptide which is specific for a variable region of an antibody combining site (ACS) of an antibody that cross-reacts with an epitope of the coronavirus, and
a physiologically acceptable carrier.

17. The method of claim 16, wherein the at least one anti-idiotypic polypeptide is a mirror image of the coronavirus epitope.

18. The method of claim 16, wherein the coronavirus is SARS-COV-2.

19. The method of claim 16, wherein the coronavirus is HCoV-HKU1, HCOV-229E, HCOV-NL63, HCoV-OC43, SARS-COV-1 or MERS-COV.
